(19) United States
(12) Patent Application Publication
(10) Pub. No.: US 2003/0050561 A1
(43) Pub. Date: Mar. 13, 2003

(54) System and method for evaluating a characteristic of body typology

(75) Inventors: Roland Bazin, Bievres (FR); Franck Giron, Ferrieres-En-Brie (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris, FR

(21) Appl. No.: 10/183,609

(22) Filed: Jun. 28, 2002

(30) Foreign Application Priority Data
Jul. 9, 2001 (FR) 0109090

(51) Int. Cl. A61B 6/00

(52) U.S. Cl. 600/476

(57) ABSTRACT

A system for evaluating a characteristic of body typology, such as a characteristic of the skin or hair appearance. The system includes a plurality of reference images that can be part of an atlas or can be displayed on a computer screen. The system also includes a portable optical instrument which produces an image of the zone under examination suitable for being compared with one of the reference images.

### CROSS-REFERENCE TO RELATED APPLICATIONS

This document claims priority to French Application No. 0109090 filed Jul. 9, 2001, the entire content of which is hereby incorporated by reference.

### BACKGROUND OF THE INVENTION

1. Field of the Invention

The present invention relates to evaluating typological characteristics of the body, in particular characteristics of appearance such as, for example, brilliance, color, and cutaneous relief.

2. Background of the Invention

Dermatoscopes with a magnifying system and integrated lighting arrangement are known. Such dermatoscopes, however, are not designed to deliver an image other than a mere magnification of the zone under observation. In addition, lighting characteristics can vary from one dermatoscope to another. This variation is not of great significance when the purpose is to observe a defect of the skin, but which is unsatisfactory when the purpose is to evaluate the characteristics of appearance such as, for example, brilliance or color.

Complex systems using video cameras or other electronic sensors are also known, as described for example in European patent application EP-A-0 655 211 and U.S. Pat. No. 5,377,000. Those systems are relatively expensive and are ill-suited for widespread distribution, e.g., for use at all points of sale of a product or to enable members of the public to perform evaluations on themselves.

### SUMMARY OF THE INVENTION

Consequently, there exists a need for a system that is simple to use, relatively inexpensive, and that enables the evaluation of characteristics of body typology, in particular characteristics of skin or hair appearance, for example, characteristics associated with the radiant nature of the complexion or hair, such as brilliance and color. One object of the present invention is to provide such as system.

The invention achieves this and other objects by providing a novel system for determining the degree of a characteristic of body typology, in particular, a characteristic of the skin and/or the hair appearance. The system includes an atlas having a plurality of reference images or a system for displaying reference images. The system also includes a portable optical instrument which enables a zone of the skin or the hair to be observed directly. The optical instrument is arranged to operate under predefined conditions of illumination to produce an image of the zone under examination. The image is suitable for being compared with one of the reference images.

Since the optical instrument of the system of the invention allows the observation to be performed directly, i.e., by looking through the optical instrument at the zone under examination, without any photographic, video, or other electronic acquisition, the optical instrument can be manufactured at a cost compatible with widespread distribution. According to a preferred embodiment of the invention, the optical instrument does not have any video system, and the image is not produced on a video screen.

Furthermore, since observation can be performed under predefined conditions of illumination, it is possible to quantify the observed characteristic in a manner that is relatively accurate and reproducible. In a particular embodiment, the optical instrument preferably includes an optical system arranged to produce a magnified image of the zone under examination. This makes it possible to evaluate characteristics of the skin that are difficult to observe with the naked eye, for example, the desquamation thereof.

In accordance with another embodiment, the optical instrument is arranged to reduce or eliminate the brilliance of at least a portion of the zone under examination. This feature facilitates the observation of, for example, the color of the skin or the hair, without being troubled by glinting or glaring reflections. Suppressing or reducing brilliance can also be useful in observing the color of backscattered light coming from deep layers of the skin or the hair because the color can depend on the state of these layers. In particular, it can be advantageous to observe the contrast between light that includes both a reflection component and a backscattered component, and light that essentially includes a backscattered component. This contrast can be achieved, for example, by illuminating the zone under examination under polarized light and by observing it with a polarization analyzer. The analyzer can include a rotary polarizer or a pair of polarizers having different polarization directions, one of which can be perpendicular to the polarization direction of the incident light and the other parallel to the incident light.

In accordance with another embodiment of the present invention, the zone under examination can be illuminated using a lighting arrangement that allows the zone to be illuminated with incident light under different angles of inclination, for example, in order to observe under diffuse lighting or under a grazing light. With the arrangement of the invention, a variety of observation conditions are thus available, making it easier to evaluate one or more particular characteristics of appearance, for example, by emphasizing the differences between two images. Grazing or glancing light can provide information concerning relief, while diffuse lighting can provide information concerning uniformity of color.

According to one embodiment, the optical instrument includes at least one screen suitable for placement between a source of light and the zone under examination to allow the zone to be illuminated only by light diffusing beneath the screen in the tissue under examination. The screen can be movable, such that it can be moved between a first position in which it is distant from a surface adjacent to the zone under examination and a second position in contact with the surface. This movable screen makes it possible to observe the skin or the hair by transillumination, i.e., by lighting a zone of the skin or the hair using light coming from adjacent zones. The skin or the hair can then function as a light guide, thus making it possible to obtain information concerning transparency. This information can be combined, where appropriate, with other information drawn from previous observations made under different lighting conditions. The screen can also be positioned so as to illuminate the zone under examination with a grazing light. The screen can have a tubular wall that, in use, extends around the zone under examination. In addition, the screen, which can have a non-circular section, can have a conical or pyramid-shaped portion that converges towards the zone under examination. Further, the optical instrument can have at least one spring configured to urge the screen into a rest position when not in use.

According to another embodiment, the optical instrument can have a reticule making it possible to measure the distance from which light diffusion is no longer visible. In accordance with another aspect of an embodiment of the present invention, the optical instrument has at least one color filter. The filter can be blue in color so as to reveal skin pigmentation, for example.

The optical instrument can also have at least one polarizer that can be placed along the path of the light between a light source and the zone under examination. The optical instrument can also have at least one polarizer placed along the path of the light between the zone under examination and an eye of the observer. The optical instrument could also include at least two polarizers of different orientations that are juxtaposed, and that are placed along the path of the light between the zone under examination and the eye of an observer. This feature makes it possible to observe the contrast between two zones of the image, and to take advantage of the rather high sensitivity of the human eye to contrast. The optical instrument can have at least one polarizer that is pivotally mounted so as to enable the user to vary the orientation of its direction of polarization relative to a reference direction. The optical instrument can have, for example, a handle incorporating an actuator member such as a knurled knob, for example, enabling the orientation of the polarizer to be varied using the same hand that holds the handle.

The optical instrument can be arranged to allow the zone under examination to be illuminated under natural light. In an exemplary embodiment, the optical instrument can include a skirt of transparent plastic material with an edge that can be placed around the zone under examination.

The optical instrument can also include at least one integrated light source. The integrated light source can include at least one light emitting element, for example, selected from the following: an incandescent lamp; a light emitting diode (LED); and a fluorescent lamp. The optical instrument can have light emitting elements that illuminate different respective wavelength ranges. By way of example, the optical instrument can include a source that reproduces the spectral characteristics of natural light, possibly including slightly-colored light emitting diodes. The optical instrument can have a plurality of light emitting elements together with a control arrangement which selectively powers at least a fraction of the light emitting elements. The optical instrument can also include light emitting elements that are disposed in a circle.

According to another embodiment, the optical instrument can have a housing for receiving one or more electrical batteries. Such a housing can have an axis substantially perpendicular to an observation direction for observing the zone under examination.

The optical instrument can include a pane which compresses the skin in the zone under examination so as to expel blood therefrom. Such a pane can be made of glass or of transparent plastic material and it can optionally be colored. Such a pane be a removable accessory or it can be an element that is permanently fixed to the optical instrument, with the pane movable between an active position in which it is interposed between the skin and the light path leading to the observer, and an inactive position in which the pane is not situated in the light path leading to the observer. According to another feature, the optical instrument can also include a ring to couple a camera to the optical instrument.

In a particular embodiment, the system includes an atlas having a plurality of reference images. These images can be placed on a single medium or they can be on media that are bound together. Each image can be associated with a respective alphanumeric indication. The system can also include a computer for displaying reference images.

The invention also provides a method of evaluating a characteristic of body typology. In a preferred embodiment, the method can include the steps of observing the skin or the hair with an optical instrument as discussed above; comparing the observed image with reference images; and selecting a reference image. In accordance with an exemplary implementation of the method, the contrast is observed between light including both a reflection component and a backscattered component and light having essentially a backscattered component. To achieve this contrast, the method can include the steps of illuminating the zone under examination with polarized light and observing it through a polarization analyzer. The reference images can be displayed on a computer screen, as mentioned above. The reference images can be transmitted to the computer from a server over a computer network, prior to being displayed on the screen.

The method can also include transmitting an indication that is representative of the selected reference image, for example, to a server. The server can then be programmed to establish a diagnosis, for example, or else to recommend a cosmetic or a care product.

In a particular implementation of the invention, a characteristic of body typology is evaluated, treatment is performed, and then a new evaluation is performed in order to detect any variation in the characteristic and determine the effectiveness of the treatment. Preferably, the treatment performed is a cosmetic treatment suitable for having an effect on the evaluated characteristic.

As mentioned above, the same optical instrument can be used to perform at least two types of observation successively or simultaneously. The observations can include:

I. observation under diffuse lighting;

II. observation under grazing light;

III. observation under directional lighting;

IV. observation by transillumination;

V. observation under polarized light using a non-crossed polarizer;

VI. observation under polarized light using a crossed polarizer;

VII. observation while compressing the skin;

VIII. observation without compressing the skin;

IX. observation while stretching the skin;

X. observation without stretching the skin;

XI. observation while creasing the skin; and

XII. observation without creasing the skin.

Particularly desirable observations are I and II, V and VI, VII and VIII, IX and X, or XI and XII, identified above. When making an observation by transillumination, it is possible to measure the distance from which light diffusion is no longer visible.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings, wherein:

FIG. 1 illustrates a fragmentary axial section view through an optical instrument for implementing the invention;

FIG. 2 is a fragmentary plan view as seen looking along arrow II of FIG. 1, showing how the lighting elements are positioned;

FIG. 3 shows the FIG. 1 device in a configuration for observation under grazing light;

FIG. 4 shows the FIG. 1 device in a configuration enabling transillumination;

FIG. 5 is a diagrammatic and fragmentary axial section view through a variant of the FIG. 1 device;

FIG. 6 shows the FIG. 5 device in a configuration providing grazing lighting;

FIG. 7 shows the FIG. 5 device in a configuration enabling transillumination;

FIG. 8 shows the polarizer of the device of FIGS. 5 to 7 in plan view;

FIG. 9 is a fragmentary plan view showing the analysis polarizer disk of the device of FIGS. 5 to 7, and also showing how the light emitters are disposed;

FIG. 10 shows a variant of the device of FIGS. 5 to 7;

FIG. 11 is a diagrammatic and fragmentary axial section view of another example of an optical instrument for implementing the invention;

FIG. 12 is a fragmentary plan view of FIG. 11;

FIG. 13 shows the FIG. 11 device in a grazing light configuration;

FIG. 14 shows the FIG. 11 device in a configuration that enables transillumination;

FIG. 15 shows another example of the device in an exploded perspective view;

FIG. 16 is a fragmentary end view seen looking along arrow XVI of FIG. 15;

FIG. 17 shows a variant of a portion of the FIG. 15 device;

FIG. 18 is a diagrammatic representation of a brilliance atlas;

FIG. 19 is a diagrammatic representation of a contrast atlas;

FIG. 20 is a diagrammatic representation of a skin dryness atlas;

FIG. 21 illustrates an atlas embodied in the form of a set of images that are bound to one another;

FIG. 22 is a diagram showing a computer enabling reference images to be displayed on a screen; and

FIG. 23 is a diagram showing a computer suitable for displaying reference images connected to a remote server according to another embodiment of the present invention.

### DETAILED DESCRIPTION OF THE PREFERRED EMBODIMENTS

Referring now to the drawings in which like referenced numbers designate like parts throughout the various views, the optical instrument 10 shown in FIGS. 1 to 4 is for observing the skin P or the hair. The arrangement includes a support 11 which is placed on the surface to be observed, an assembly 12 movable relative to the support 11 along an axis X, and a handle 13 that allows the instrument to be held by a user. In a preferred embodiment, the handle 13 is fixed relative to the support 11 and can include one or more optionally rechargeable batteries 14.

A resilient return system acts between the support 11 and the movable assembly 12 so as to urge the assembly 12 toward a rest position corresponding to FIG. 1 in which the assembly 12 is pressed against the handle 13. In the example shown, this return system includes a plurality of springs 15 working in compression, with each having one end bearing against a shoulder of the support 11 and an opposite end bearing against the assembly 12.

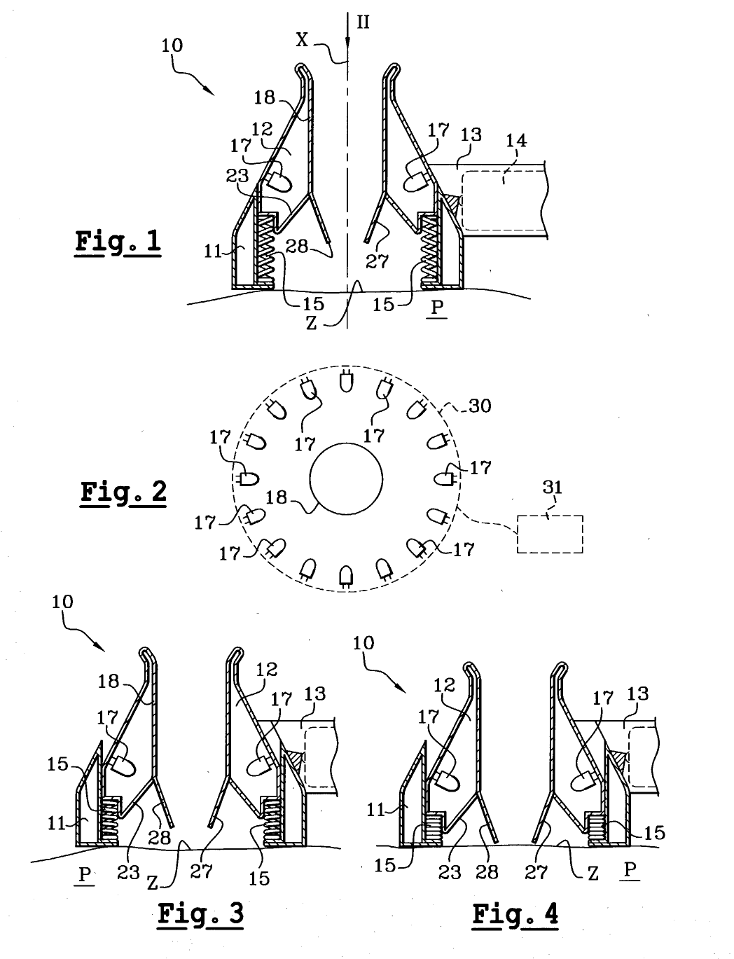

The assembly 12 can include a plurality of light emitters 17. In the example illustrated white light emitting diodes are distributed all around the axis X, as shown in FIG. 2, so as to produce either diffuse lighting of the zone Z under examination when all of the light emitters are powered, or lighting in one or more particular directions, when only a fraction of the light emitters are powered. The light emitters 17 can be powered electrically, for example, by the batteries 14 via a power supply circuit 30, which is represented diagrammatically or schematically for clarity. The power supply circuit 30 is connected to a control circuit 31. Observation is performed via a tube 18 of the moving assembly 12.

An optical element 23 is placed on the path of light between the light emitters 17 and the zone Z under examination. In the example shown, this optical element 23 includes a translucent annular piece which can be colorless or colored. This optical element can function as a diffuser. The tube 18 is extended downwards by a screen 27 having a free edge 28, which defines the field of observation. The optical instrument 10 enables the same surface to be observed under different lighting conditions in succession.

In the configuration shown in FIG. 1, the zone Z under examination can be illuminated in diffuse and omnidirectional manner by all of the light emitters 17. The screen 27 can be moved towards the zone Z under examination because the assembly 12 is movable relative to the support 11, as shown in FIG. 3. As seen in FIG. 3, the bottom edge 28 of the screen 27 has moved by exerting sufficient downward pressure on the assembly 12 to overcome the action of the return springs 15. This feature enables the zone Z under examination to be illuminated with grazing light, which more clearly reveals the relief of the skin or the hair and increases brilliance.

The user also has the option of pressing fully against the assembly 12 so as to bring the screen 27 into contact with the surface of the skin or the hair, as shown in FIG. 4. Under this configuration, the zone Z under examination is illuminated solely by transillumination, i.e., by light diffusing through the tissue under observation. This configuration provides information concerning the transparency of the tissue, its color, and the extent to which it is irrigated by blood.

The electrical power delivered to the light emitters 17 can remain unchanged between the various observations. The control circuit 31 can include merely an electrical switch providing on/off control of power delivery to all of the light emitters 17, or it can be more complex. For example, the circuit 31 can power only some of the light emitters 17 in selective manner in order to obtain directional or pluridirectional lighting that can be stationary or rotating. If the light emitters are LEDs of different colors or capable of emitting various different wavelengths, the circuit 31 can modify the spectral characteristics of the light illuminating the zone under examination.

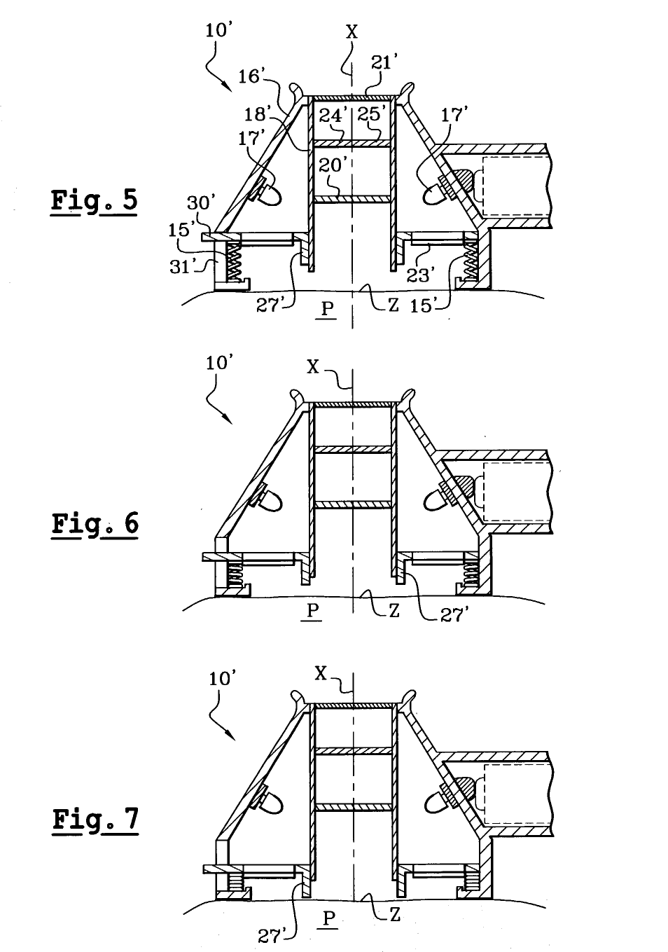

FIGS. 5 to 7 show an optical instrument 10′ according to another embodiment of the present invention. In this embodiment, the instrument 10′ includes a body 16′ which is stationary and a screen 27′ which is movable relative to the body 16′ along the axis X in order to modify the lighting conditions applied to the zone Z under observation. For example, the lighting conditions can be switched between multidirectional diffuse lighting and grazing light, as shown in FIG. 6, and lighting by transillumination, as shown in FIG. 7.

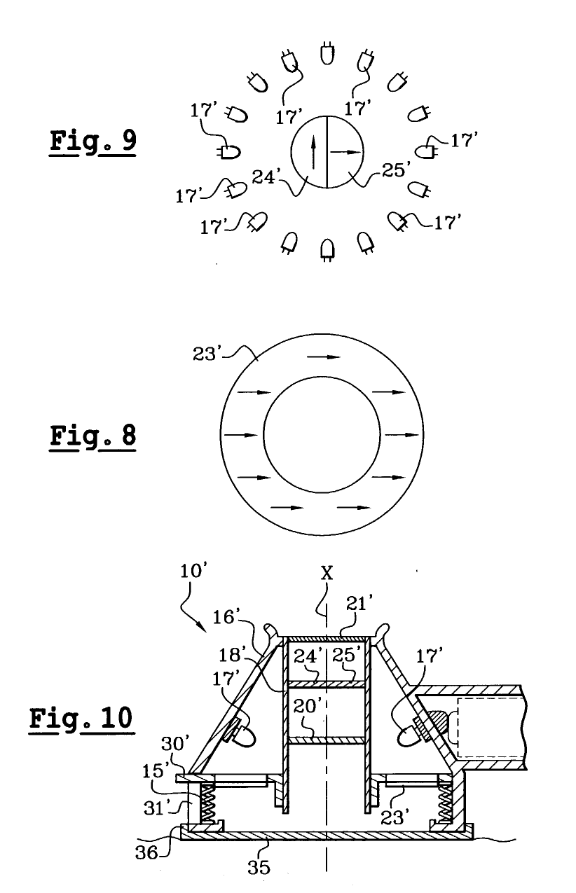

A polarizer 23′ shown on its own in FIG. 8 is placed on the path of the light from the light emitters 17′ and the zone Z under observation. This polarizer 23′ can be annular in shape and can provide polarization in a single direction, as represented by arrows in FIG. 8. Of course, the polarizer 23′ can have other shapes and other polarization.

The screen 27′ is mounted to slide on a tube 18′. The tube 18′ supports lenses 20′ and 21′, and polarizers 24′ and 25′. The screen 27′ can be moved using a tab 30′ which projects out from the instrument through a slot 31′ in the body 16′ on which the light emitters 17′ are mounted. The light emitters 17′ can be arranged in the same manner as the light emitters 17 in the assembly of FIGS. 1-4. An advantage of the instrument 10′ is that, while changing the lighting configuration by moving the screen 27′, the distance between the zone under examination and the lenses 20′, 21 ′ does not change.

In the example under consideration, each of the two polarizers 24′ and 25′ is in the form of a half-disk. The two half-disks are connected together via their bases so as to form a single disk for polarization analysis. This disk is placed on the light path in the tube 18′ in the image focal plane of the lens 20′. The polarization directions of the polarizers 24′ and 25′ are perpendicular to each other and are represented by the arrows in FIG. 9.

The polarization analyzer including the polarizers 24′ and 25′ provides one half-image with brilliance and another half-image without brilliance by selecting the direction of polarization of one of the half-disks 24′ and 25′ to be parallel to the polarization direction of the polarizer 23′. Where appropriate, the polarizers 24′ and 25′ are replaced by a single polarizer mounted to turn about the axis X so as to be capable firstly of making its polarization direction coincide with the polarization direction of the incident light and secondly of making its direction of polarization extend perpendicularly to the polarization direction of the polarizer 23′. The two-part polarization analyzer 24′, 25′ provides an image that is contrasted, so as to take advantage of the high sensitivity of the human eye to contrast.

As shown in FIG. 10, each instrument 10 or 10′ can include a pane 35 through which observation is performed. This pane 35 can be made of a plastic material. The pane 35 can have, for example, a rim 36 enabling the pane 35 to be put into position on the instrument 10 and 10′. The pane 35 can be a removable accessory or a retractable accessory, remaining secured to the instrument in its retracted position. When in place on the instrument, the pane 35 compresses the skin and expels blood from it. The pane allows observations on the skin while reducing the incidence of blood color on skin color. It is possible to perform one series of observations without the pane 35 and another series of operations with the pane 35 under various types of lighting or polarization, and then to compare the results to extract useful information therefrom.

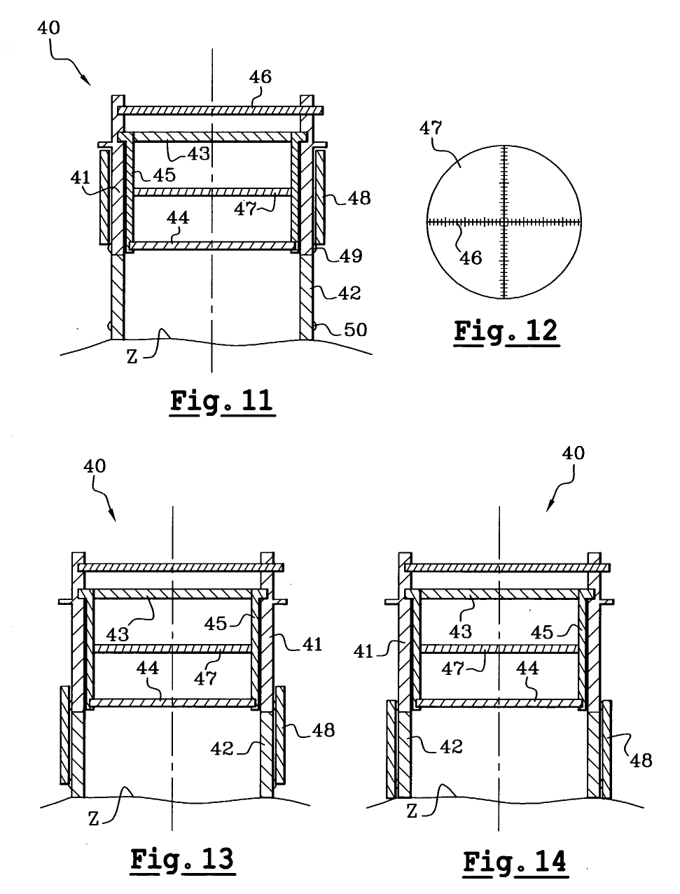

Compared with the instruments 10 and 10′ described above, FIGS. 11, 13, and 14 show an instrument 40 without integrated lighting. Thus, while these embodiments provide arrangements for exposing the zone under examination with light, the structure of the instrument need not itself include a light source, as the light source can be provided separately. The instrument 40 includes a body 41 which is extended downwards by a transparent skirt 42. The body 41 serves as a support for an optical assembly including lenses 43 and 44 carried by a tube 45 and snap-fastened in the body 41. The optical assembly also includes a reticule 46 enabling a distance to be measured on the zone under examination. By way of example, FIG. 12 shows a reticule printed on a glass plate 47 placed on the path of light coming from the zone under examination. A color filter 46 is removably mounted in the body 41. In the example shown, the filter is blue so as to emphasize, if so desired, spots of pigmentation.

A screen constituted by an opaque ring 48 is slidably mounted on the body 41 and the transparent skirt 42. The ring 48 moves between a fully retracted position corresponding to FIG. 11 in which the ring 48 does not cover the transparent skirt 42, an intermediate position corresponding to FIG. 13, in which the ring 48 covers a major fraction of the transparent skirt 42 so as to allow grazing light only on the zone under examination, and a position in which the ring 48 is fully lowered as shown in FIG. 14 where the ring covers the entire transparent skirt 42 and enables illumination to be performed by transillumination.

In the example shown, projections 49 and 50 are formed on the body 41 and the transparent skirt 42 so as to enable the skirt 48 to be held stationary in each of the above-described configurations. Other positioning mechanisms can be used so as to position the screen 48 at various positions along the skirt 42. These positions are not intended to be limited to three or some other number, but can be any position between the fully retracted position and the fully covered position. When the zone under examination is observed by transillumination, the observer can use the reticule 46 to measure the distance beyond which light diffusion is no longer visible, and thereby gain information concerning the transparency of the skin.

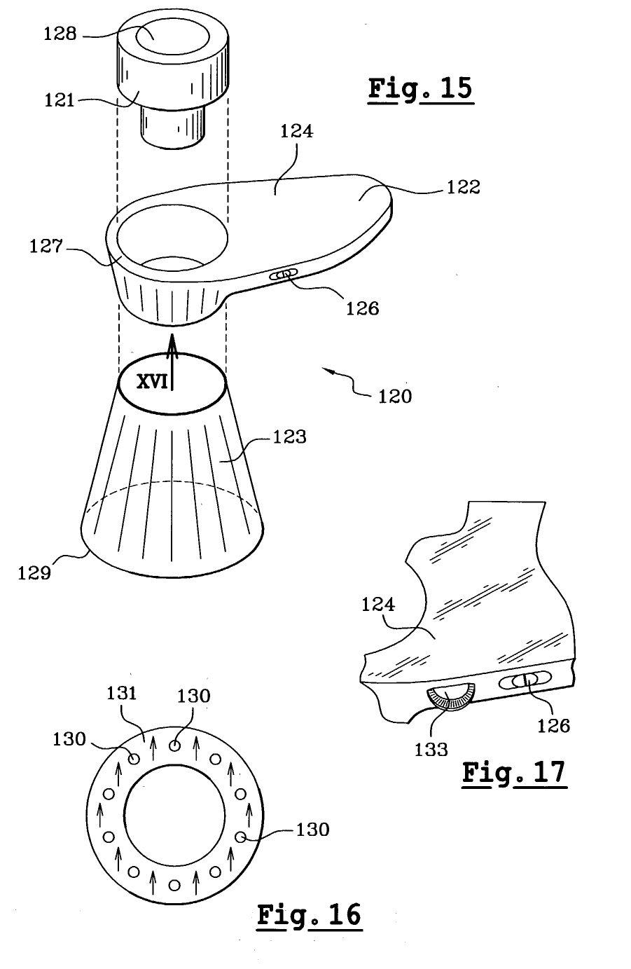

FIG. 15 shows another embodiment of an observation device 120 made in accordance with the invention. The device 120 includes a light source 122, an endpiece 123, and an optical assembly 121. The light source 122 includes a handle 124 forming a housing containing one or more optionally rechargeable batteries, and an open portion 127 in which the optical assembly 121 can be engaged.

The optical assembly includes an eyepiece 128 having one or more lenses (not shown) so as to produce a magnified image, together with a polarizer. The light source 122 includes a selector 126 configured to control the light emitted by the light source 122. For example, the selector 126 can be configured to control the light intensity so that the zone for observation can be illuminated with two different light intensities. The selector 126 can also be configured to control the type of light so that the zone for observation can be illuminated with two or more different types of lighting, e.g. one type simulating daylight and another type simulating incandescent lighting.

By way of example, the endpiece 123 is preferably frustoconical in shape having a base 129 of diameter greater than the field of observation of the optical assembly 121. In the example shown, the diameter of the base 129 is about 40 millimeters (mm) and that of the field of observation is about 30 mm, of course other dimensions are within the scope of the present invention. The dimensions can be designed to reduce or avoid contact pressure between the endpiece 123 and the skin having an influence on the appearance of the zone situated in the field of observation. It is possible to provide magnetic fixing between the endpiece 123 and the light source 122. In addition, by way of example, the endpiece 123 can include a magnetized metal ring. At its end which comes into contact with the skin, the endpiece 123 can include a removable ring.

As can be seen in FIG. 16, the light source 122 includes a plurality of light sources 130. For example, the light sources can be light emitting diodes 130. Each of the diodes 130 can be positioned on an axis substantially parallel to that of the optical assembly 121. The light emitting diodes 130 can be covered by a polarizer 131. The height of the endpiece 123 can be selected so that the illumination on the zone of the skin situated in the field of observation is substantially uniform. As shown in FIG. 17, the device 120 can include a knurled knob 133 enabling the user to turn the optical assembly 121 with the same hand as is used for holding the handle 124.

The observation device 120 can be used as discussed next. The endpiece 123 and the optical assembly 121 can be put into place on the light source 122, after which the user can turn the optical assembly 121 relative to the light source so that the polarizer contained in the optical assembly 121 has the same polarization direction as the polarizer 131 or has a direction that is substantially perpendicular thereto. This makes it possible to observe the skin successively with brilliance and without brilliance. In another embodiment, the optical assembly 121 has two juxtaposed polarizers with directions of polarization that are perpendicular, similar to the example described above with reference to FIG. 9.

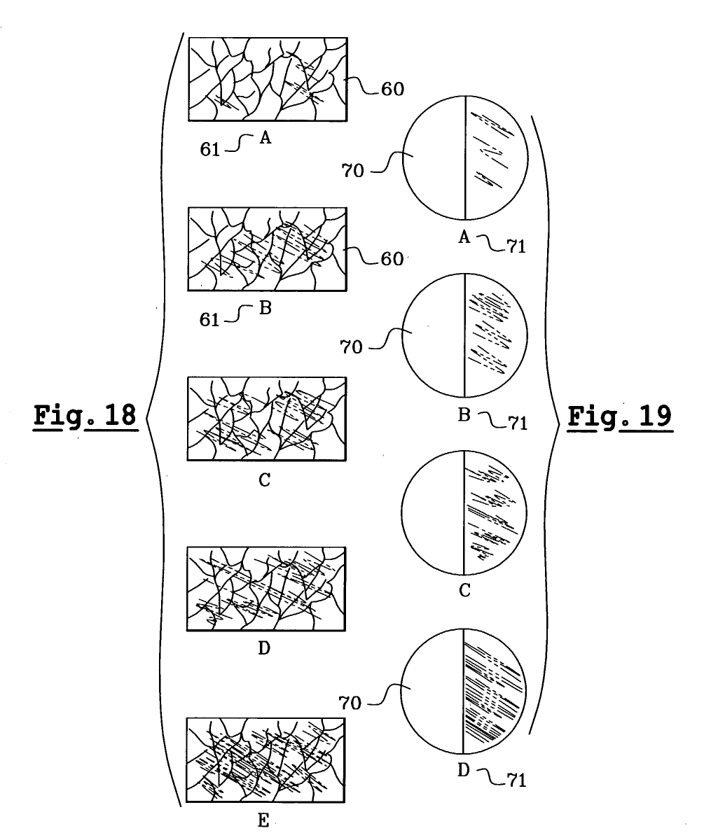

Each image observed using one of the optical instruments described above, can be compared with a reference image of an atlas including a plurality of reference images. For example, the atlas can include images expressing varying degrees of different characteristics of body typology, in particular the brilliance or the color of the skin, or images corresponding to various degrees of contrast. FIG. 18 is a diagrammatic representation of a brilliance atlas including a plurality of reference images 60 each corresponding to a different degree of skin brilliance, and quantified by an alphanumeric identifier 61.

FIG. 19 is a diagrammatic representation of a contrast atlas, i.e. showing differences in luminosity or color, including a plurality of reference images 70, corresponding to various degrees of contrast or difference that are likely to be observed when using a polarization analyzer of the kind formed by the half-disks 24′, 25′ as described above. These images 70 are associated with alphanumeric identifiers 71 enabling them to be identified.

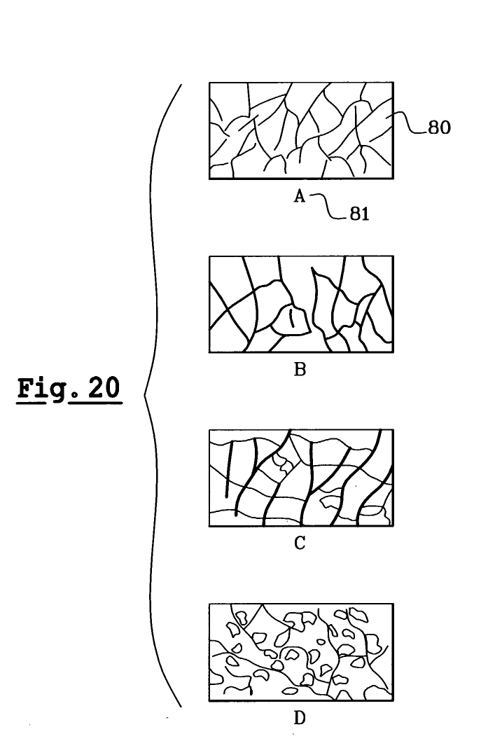

FIG. 20 is a diagrammatic representation of an atlas of skin dryness, including a plurality of images 80 each associated with an alphanumeric identifier 81. The images 80 express various degrees of skin dryness, going from severe desquamation, characteristic of extremely dry skin, to no desquamation, representing normal skin.

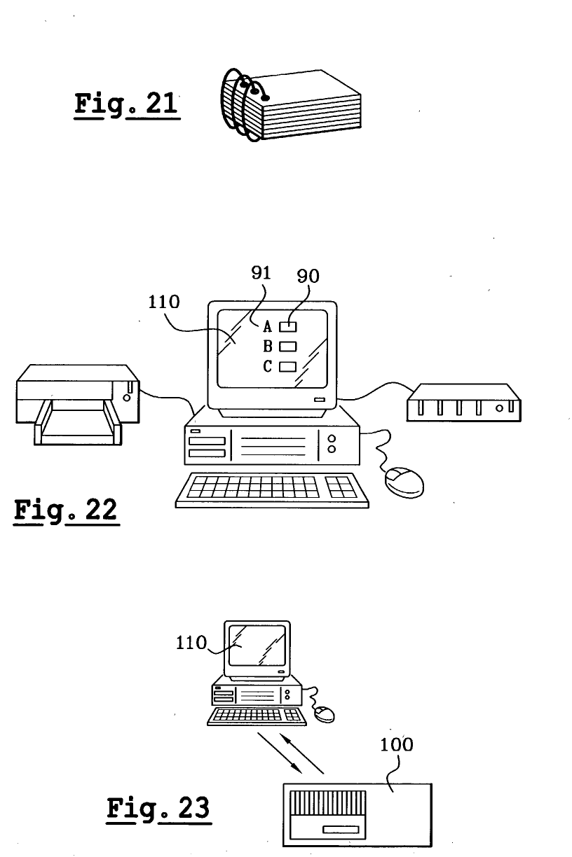

The reference images in an atlas can be printed on a common medium, or a plurality of media can be bound together, as shown in FIG. 21. Reference images can also be displayed on the screen of a computer 110, as shown in FIG. 22, each image being associated with an alphanumeric identifier. The results of an evaluation can be transmitted remotely to a server 100 over a computer network, in particular the Internet, from the computer 110, as shown in FIG. 23. The server 100 can be configured to provide a diagnosis as a function of the results transmitted to it, and where appropriate to recommend a cosmetic or care product.

When the reference images are displayed on the screen of a computer, these images can be transmitted by the server 100 after the user has connected to the corresponding Internet site. The invention can be implemented in such a manner as to track the effectiveness of a course of treatment, with evaluation being performed after each stage of treatment, and with the results of successive evaluations being compared.

Because of the possibility of using a single optical instrument to obtain information concerning brilliance, color, relief, and transparency, and by using an atlas that enables such information to be quantified, the present invention makes it possible to create a multi-vector databank bringing together vectors each corresponding to a particular individual, each vector having at least two components each constituted by the result of an observation performed using the same optical instrument.

Naturally, the invention is not limited to the examples described above and the structure of the optical instrument can be modified, in particular by replacing the light emitting diodes with other lighting sources, such as incandescent lamps or fluorescent lamps, for example. When a screen is used for observation by transillumination, the screen can be constituted by a separate element that is releasably applied to the optical instrument.

Obviously, numerous modifications and variations of the present invention are possible in light of the above teachings. It is therefore to be understood that within the scope of the appended claims, the invention may be practiced otherwise than as specifically described therein.

What is claim as new and desired to be secured by Letters Patent of the United States is:

1. A system for evaluating a characteristic of body typology, comprising:
at least one of (a) an atlas having a plurality of reference images, and (b) means for displaying a plurality of reference images; and
a portable optical instrument which enables a zone of a body to be observed directly and which produces an image of said zone, said image being suitable for a comparison with one of said reference images.

2. A system according to claim 1, wherein said system includes said atlas of said plurality of references.

3. A system according to claim 1, wherein said system includes said means for displaying said plurality references.

4. A system according to claim 1, wherein said optical instrument is arranged to produce a magnified image of the zone under examination.

5. A system according to claim 1, wherein the optical instrument is arranged to expose the zone under examination to diffuse lighting.

6. A system according to claim 1, wherein the optical instrument is arranged to expose the zone under examination to directional lighting.

7. A system according to claim 1, wherein the optical instrument is arranged to expose the zone under examination to grazing light.

8. A system according to claim 1, wherein the optical instrument comprises at least one screen suitable for being interposed between a light source and the zone under examination.

9. A system according to claim 8, wherein said screen exposes the zone under examination to lighting by transillumination.

10. A system according to claim 8, wherein the screen is movable from a first position at which the screen is remote from a surface surrounding the zone under examination and a second position at which the screen comes into contact with said surface.

11. A system according to claim 8, wherein the screen is movable to a position so that the zone under examination is illuminated by grazing light.

12. A system according to claim 8, wherein the screen comprises a tubular wall extending around the zone under examination.

13. A system according to claim 12, wherein the screen comprises a portion that is conical or pyramid-shaped, converging towards the zone under examination.

14. A system according to claim 8, wherein the screen is movable, and wherein the optical instrument comprises at least one spring which urges the screen toward a rest position.

15. A system according to claim 1, wherein the optical instrument comprises at least one color filter.

16. A system according to claim 15, wherein the filter has a blue color.

17. A system according to claim 1, wherein the optical instrument comprises at least one polarizer.

18. A system according to claim 17, wherein the optical instrument comprises at least one polarizer placed on the path of light between a light source and the zone under examination.

19. A system according to claim 17, wherein the optical instrument comprises at least one polarizer placed on the path of the light between the zone under examination and an observer's eye.

20. A system according to claim 1, wherein the optical instrument comprises at least two polarizers of different orientations that are juxtaposed and placed on the path of light between the zone under examination and an observer's eye.

21. A system according to claim 1, wherein the optical instrument comprises at least one pivotally-mounted polarizer so as to enable a user to modify the orientation of its direction of polarization relative to a reference direction.

22. A system according to claim 1, wherein the optical instrument is arranged to expose the zone under examination to natural light.

23. A system according to claim 22, wherein the optical instrument comprises a skirt of transparent plastic material, the skirt having an edge extending around said zone under examination.

24. A system according to claim 1, wherein the optical instrument comprises at least one integrated light source.

25. A system according to claim 24, wherein the integrated light source comprises at least one light emitting element selected from the group consisting of an incandescent lamp, a light emitting diode, and a fluorescent lamp.

26. A system according to claim 25, wherein the optical instrument comprises light emitting elements emitting light in respective different wavelength ranges.

27. A system according to claim 24, wherein the optical instrument comprises a plurality of light emitting elements and control means for selectively powering at least a fraction of said light emitting elements.

28. A system according to claim 24, wherein the optical instrument comprises light emitting elements disposed in a circle.

29. A system according to claim 24, wherein the optical instrument comprises a housing for enclosing one or more electric batteries.

30. A system according to claim 29, wherein the housing has an axis substantially perpendicular to an observation direction for observing the zone under examination.

31. A system according to claim 1, wherein the optical instrument comprises a pane which compresses the skin in the zone under examination so as to expel blood therefrom.

32. A system according to claim 31, wherein said pane is removable.

33. A system according to claim 32, wherein the reference images are placed on a single medium.

34. A system according to claim 1, wherein each of said reference images is associated with an alphanumeric indication.

35. A system according to claim 3, wherein the means for displaying said reference images comprises a computer.

36. A system according to claim 1, wherein the optical instrument comprises a reticule.

37. A method of evaluating a characteristic of a body, comprising the steps of:
observing an image of skin or hair with the system of claim 1;
comparing the observed image with said reference images; and
selecting one of said reference images.

38. A method according to claim 37, wherein the step of observing comprises observing a contrast between light which comprises both a reflected component and a backscattered component and light which essentially comprises a backscattered component.

39. A method according to claim 38, further comprising the steps of:
illuminating the zone under examination with polarized light; and
observing said zone with a polarization analyzer.

40. A method according to claim 37, further comprising displaying the reference images on a screen of a computer.

41. A method according to claim 40, further comprising transmitting said reference images to the computer from a server over a computer network prior to the displaying step.

42. A method according to claim 41, further comprising the step of transmitting to the server an indication representative of the selected reference image.

43. A method according to claim 37, wherein a same optical instrument is used successively or simultaneously to perform at least two types of observation selected from the group consisting of:
observation under diffuse lighting;
observation under grazing light;
observation under directional lighting;
observation by transillumination;
observation under polarized light using a non-crossed polarizer;
observation under polarized light using a crossed polarizer;
observation while compressing the skin;
observation without compressing the skin;
observation while stretching the skin;
observation without stretching the skin;
observation while creasing the skin; and
observation without creasing the skin.

44. A method according to claim 37, further comprising the steps of performing a cosmetic treatment; and
observing another image of said skin or hair in order to detect any change in said characteristic and to determine the effectiveness of the cosmetic treatment.

45. A method according to claim 43, wherein the step of observing is performed by transillumination and further comprising the step of measuring a distance beyond which light diffusion is no longer visible.

46. A system for evaluating a characteristic of body typology, comprising:
a portable device which provides an image of a zone of a body; and
a plurality of reference images expressing varying degrees of said characteristic of body typology.

47. A system of claim 46, wherein said reference images express varying degrees of skin brilliance.

48. A system of claim 46, wherein said reference images express varying degrees of skin color.

49. A system of claim 46, wherein said reference images express varying degrees of skin dryness.

50. A system of claim 46, wherein said reference images express varying degrees of hair brilliance.

51. A system of claim 46, wherein said reference images express varying degrees of hair color.

52. A system of claim 46, wherein said reference images are contrast images.

53. A system of claim 46, wherein said reference images are grouped in an atlas.

54. A system of claim 46, wherein said reference images are stored in a memory.

55. A system of claim 54, further comprising a computer comprising said memory and which displays said reference images on a screen.

56. A system of claim 54, further comprising a computer network comprising a computer and a server, wherein said server comprises said memory and is connected to said computer.

57. A system of claim 56, wherein:
said network transmits said reference images to said computer; and
said computer displays said reference images.

58. A system of claim 57, wherein said computer transmits a result of a comparison between said image of said zone and said reference images to said network.

59. A system of claim 58, wherein said network transmits a diagnostic to said computer based on said result.

60. A system of claim 58, wherein said server transmits a recommendation for a cosmetic product to said computer.
